SEQUENCE LISTING (5 sequences)

SEQ ID NO: 1 - protein, 22 aa, Homo sapiens
ATGLALSSLRLRNVSWATGRSW

SEQ ID NO: 2 - protein, 12 aa, Homo sapiens
ALSSLRLRNVSW

SEQ ID NO: 3 - protein, 12 aa, Homo sapiens
LSCNRLNRAPQP

SEQ ID NO: 4 - protein, 22 aa, Homo sapiens
ALGTSLARLSNRLWSVGTAWSR

SEQ ID NO: 5 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

(19) United States
(12) Patent Application Publication
(10) Pub. No.: US 2010/0331239 A1
(43) Pub. Date: Dec. 30, 2010

(54) CD14 AND PEPTIDES THEREOF FOR PROTECTION OF CELLS AGAINST CELL DEATH

(75) Inventors: Boris Tartakovsky, Tel Aviv (IL); Elizabeth Naparstek, Jerusalem (IL)

Correspondence Address: THE NATH LAW GROUP, 112 South West Street, Alexandria, VA, 22314, US

(73) Assignees: THE MEDICAL RESEARCH, INFRASTRUCTURE AND HEALTH SERVICES; FUND OF THE TEL AVIV MEDICAL CENTER, Tel Aviv (IL)

(21) Appl. No.: 12/864,940

(22) PCT Filed: Jan. 29, 2009
(86) PCT No.: PCT/IL09/00113

§ 371 Date: Aug. 31, 2010

Related U.S. Application Data
(60) Provisional application No. 61/006,717, filed on Jan. 29, 2008.

(51) Int. Cl. A61K 38/17; C07K 7/08; C07K 14/705; C12Q 1/06; A61K 38/10; A61P 25/00; A61P 9/00; A61P 35/00; A61P 31/00; A61P 37/02; C12N 5/078

(52) U.S. Cl. 514/2.3; 530/327; 530/326; 514/18.9; 435/39; 514/21.2; 514/21.4; 514/21.5; 514/17.7; 514/16.4; 514/19.3; 435/375

(57) ABSTRACT

Provided is a method of using soluble CD14 or peptides derived therefrom for protecting cells from death, specifically from apoptotic cell death. Further provided are compositions including CD14 or CD14 peptides and methods for protecting cells, in particular lymphocytes, from apoptotic cell death. The compositions and methods are relevant, in particular, for the treatment of various immune deficiencies associated, for example, with cell transplantation procedures, autoimmune diseases and infectious diseases such as HIV.

### FIELD OF THE INVENTION

This invention relates to therapeutic compounds capable of protecting cells from death. In particular, the present invention relates to CD14 or fragments thereof, as well as to pharmaceutical compositions, and methods for treating disorders associated with cell death.

### BACKGROUND OF THE INVENTION

CD14 is a 55-kDa glycosylphosphatidylinositol (GPI)-linked protein present on the surface membrane of phagocytic leukocytes. It is also present in a soluble form in serum. CD14 is one of the major molecules responsible for the innate host inflammatory response to microbial infection. As a key receptor for lipopolysaccharide (LPS) on the surface of monocytes and macrophages, the CD14 molecule was thought, until recently, to be involved primarily in non-specific host defense mechanisms against gram-negative bacteria (1-5). However, it is becoming clearer that non-myeloid cells also express CD14 although its function in these cells is still obscure (6).

A number of recently published results confer to this interesting molecule novel functions that are linked to apoptosis and also to T-cell activation. Thus, CD14 may function as an “apoptotic cell-receptor” on the surface of phagocytes since it seems to bind to structures which are externally exposed by apoptotic cells (7, 8); it may also be linked to susceptibility of monocytes to apoptosis as it has been shown that a high level of expression of the membrane form of CD14 correlates with monocyte resistance to apoptosis, and vice-versa (9, 10). Expression of membrane-bound CD14 was also demonstrated on the apical surface of enterocytes. Yu et al (16) suggested that membrane-bound CD14 is involved in caspase dependent activities which lead to enterocyte rescue from LPS-induced apoptosis.

CD14 either as a recombinant protein or as a native molecule secreted by monocytes in vitro has been shown to bind to the surface of in vitro activated human T cells (11, 12). This binding was shown to convey a negative signal onto these T cells (13), in the form of IL2, IL4, and IFN gamma inhibition, probably due to the inactivation of NFκB.

The inventor of the present invention has identified a subpopulation of lymphocytes in which a CD14-like antigen could be visualized by immunofluorescence but only upon fixation and permeabilization of cells, implying that this molecule was located intracellularly and not on the outer membrane of the cells (14). This intracellular antigen could be detected almost exclusively by the MO2 monoclonal antibody, an antibody directed against human CD14.

### SUMMARY OF THE INVENTION

The present invention is based on the surprising finding that soluble CD14 protects cells from death, specifically from apoptotic cell death. Moreover, a peptide derived from CD14 (a CD14 peptide) was also found to have anti-apoptotic activity, similar to the complete molecule.

Accordingly, by a first of its aspects the present invention provides use of a polypeptide having at least 80% amino acid sequence identity to the amino acid sequence of the soluble CD14 polypeptide (SEQ ID NO: 5), a fragment thereof or a peptide derived there from which retain the protective activity of the complete CD14 polypeptide, for protecting cells against cell death.

In one embodiment, the present invention provides use of a soluble CD14 polypeptide (SEQ ID NO: 5), for protecting cells against cell death.

In another aspect the present invention provides use of at least one peptide derived from CD14 (a CD14 peptide) which exhibits protective activity, for protecting cells against cell death.

In one specific embodiment, said CD14 peptide comprises the amino acid sequence ATGLALSSLRLRNVSWATGRSW (SEQ ID NO: 1) and is termed herein the MO2 peptide (pMO2).

In another embodiment, said CD14 peptide comprises the amino acid sequence LSCNRLNRAPQP (SEQ ID NO: 3) and is termed herein pMO2 II.

Also encompassed by the invention are any variants of pMO2 or pMO2II.

In another embodiment, the present invention provides use of a combination of at least two CD14 peptides which exhibit protective activity, for protecting cells against cell death. For example, use of a combination of the pMO2 and pMO2II peptides (SEQ ID NOS 1 and 3).

In the context of the present invention CD14 and CD14 peptides are used for protecting cells against death caused by any mechanism including, for example, necrosis or autophagy. In a preferred embodiment CD14 and CD14 peptides are used for protecting cells against apoptotic cell death, whereby the protective activity is an anti-apoptotic activity.

In another aspect, the present invention provides isolated CD14 peptides which exhibit protective activity against cell death. In a specific embodiment said CD14 peptide comprises pMO2 (SEQ ID NO. 1), and variants thereof. In another specific embodiment said CD14 peptide comprises pMO2II (SEQ ID NO: 3) and variants thereof.

The soluble CD14 and CD14 peptides are of a mammalian source, preferably, human soluble CD14 or human CD14 peptides.

The invention also contemplates pharmaceutical compositions comprising as an active ingredient soluble CD14 or fragments thereof and a pharmaceutically acceptable carrier. The invention also concerns pharmaceutical compositions comprising as an active ingredient at least one CD14 peptide or any variant thereof, and a pharmaceutically acceptable carrier. In one specific embodiment said CD14 peptide is pMO2. In another specific embodiment said CD14 peptide is pMO2II. In another embodiment said pharmaceutical composition comprises a combination of at least two CD14 peptides. In one embodiment said peptides are pMO2 and pMO2II.

The present invention also encompasses use of soluble CD14, CD14 peptides or variants thereof in the preparation of a pharmaceutical composition. In one specific embodiment said CD14 peptide is pMO2. In another embodiment said CD14 is pMO2II.

Preferably, the pharmaceutical compositions are used for inhibiting cell death, more preferably, apoptotic cell death.

The pharmaceutical compositions are also useful in the treatment of a variety of conditions involving apoptotic cell death. Examples include hematopoietic stem cell (HSC) transplantation, immune deficiencies, infectious diseases (e.g. HIV), autoimmune diseases, central nervous system (CNS) disorders, heart diseases, and aging.

According to another aspect the present invention provides a method for protecting cells against cell death comprising contacting the cells with a soluble CD14 polypeptide or a fragment thereof which retains the protective activity of the complete CD14 polypeptide.

In another aspect the present invention provides a method for protecting cells against cell death comprising contacting the cells with at least one peptide derived from CD14 (a CD14 peptide), which exhibits protective activity against cell death. In one specific embodiment said CD14 peptide is pMO2. In another specific embodiment said CD14 peptide is pMO2II. In another embodiment said method comprises administering a combination of at least two CD14 peptides. In one embodiment said peptides are pMO2 and pMO2II.

In one embodiment, the method of the invention is performed in vitro.

In another embodiment the method of the invention is performed in vivo.

In other embodiments the present invention provides a method for protecting cell types selected from a group consisting of hematopoietic cells (e.g. neutrophils), epithelial cells, stem cells, central and peripheral nervous system cells (such as neurons and glial cells, e.g. astrocytes), fibroblasts, endothelial cells, and other cell types. In a specific embodiment the present invention provides a method for protecting lymphocytes against apoptosis.

In accordance with the invention, the apoptosis of the cells may result from any condition or agent that is known in the art to induce programmed cell death.

In addition to the forgoing compositions, the invention provides methods for treating and/or preventing various medical conditions associated with apoptotic cell death. Examples of conditions suitable for treatment and/or prevention according to the methods of the invention include but are not limited to hematopoietic stem cell (HSC) transplantation, immune deficiencies, infectious diseases (including for example, HIV), autoimmune diseases, central nervous system (CNS) disorders, heart diseases, aging, and cancer, including apoptosis induced by chemotherapy side effects, for example apoptosis of gastrointestinal epithelial cells.

The compositions of the invention may also protect lymphocytes with anti-tumor activity, such as cytotoxic T cells and NK cells, from apoptosis, and thereby serve as anti cancer therapeutics.

The methods generally comprise administering to a subject in need thereof a therapeutically effective amount of CD14, at least one of the anti apoptotic CD14 peptides of the invention, or pharmaceutical compositions comprising same. Soluble CD14 or the anti apoptotic peptide(s) may also be administered in combination with one or more further therapeutic agents. The subject of the methods of the invention may be a mammal, preferably a human.

In another aspect, the present invention provides a method of determining the apoptotic status of cell (in particular lymphocytes) in patients comprising measuring the amount, or percentage of the total, of lymphocytes having internally expressed (as opposed to membrane associated) MO2 (hereinafter MO2 positive lymphocytes) whereby MO2 positive lymphocytes represent the apoptosis-protected lymphocyte population.

In another aspect the present invention provides a method of treating diseases in which apoptosis is desirable, e.g. cell proliferative diseases such as cancer, comprising administering to a patient in need thereof a therapeutically effective amount of an antagonist of CD14. Said antagonist may be for example, an antibody, or a siRNA molecule.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graphical representation of the amino acid sequence of the human CD14 showing the location and sequence of pMO2, and the minor MO2 epitope (pMO2II). The sequence of the scrambled peptide pSCR is also shown.

FIG. 2 is an inverted microscope photograph showing human mononuclear cell cultures grown overnight in: A—medium only; B—medium supplemented with 0.1 μg/ml Gliotoxin and the diluent of rhCD14; C—medium supplemented with 0.1 μg/ml Gliotoxin and 10 μg/ml rhCD14.

FIG. 3 is a graph showing a fluorescence activated cell sorter (FACS) analysis of the CD3 positive cells shown in FIG. 2. A—Cell size measured by Forward Scatter. B—Dioc6 (3) binding measurement.

FIG. 4 is a graph showing the increase in the amount of live cells, and the decrease in the amount of apoptotic cells in cultures treated with gliotoxin+CD14 (Glio+rCD14), compared to gliotoxin alone (Glio). Cells grown in medium served as a control. Apoptosis is assayed by measuring 7AAD staining.

FIG. 5 is a graph showing the percentage of apoptotic cells in cultures treated with gliotoxin+CD14, compared to gliotoxin alone. Cells grown in medium served as a control. Apoptosis is assayed by measuring DioC6(3) staining. Numbers represent mean values of 5 different experiments, with SD.

FIG. 6 is a graph demonstrating titration of rhCD14. Apoptosis of CD3+ cells was determined by Dioc6 (3) staining.

FIG. 7 is a graph demonstrating titration of rhCD14. Apoptosis of CD3+ cells was determined by measuring 7AAD binding.

FIG. 8 is a graph showing the amount of live cells, and apoptotic cells in lymphocyte cultures treated with gliotoxin (Glio), gliotoxin+CD14 (Glio+rCD14), and gliotoxin+CD14+anti CD14 antibodies (Glio+rCD14+AntiCD14) as assayed by measuring cell size (A) or DioC6(3) staining (B).

FIG. 9 is a graph showing the amount of live cells, and apoptotic cells in CD3+ lymphocyte cultures treated with gliotoxin+CD14 (Glio+rCD14), gliotoxin+CD14+anti CD14 antibodies (Glio+rCD14+AntiCD14), and gliotoxin+CD14+anti actin antibodies (Glio+rCD14+Anti-actin) as assayed by measuring cell size (A) or DioC6(3) staining (B). The protective effect of the human recombinant CD14 is completely neutralized by the monoclonal anti-CD14 antibody but not by the monoclonal anti-actin antibody of same isotype.

FIG. 10 is a graph demonstrating neutralization of the anti apoptotic effect of rhCD14 (10 μg/ml) by monoclonal anti CD14 antibodies. Monoclonal anti actin antibodies served as a control. The graph shows a summary of three different experiments.

FIG. 11 is a graph showing the percentage of apoptotic cells in CD3+ cultures treated with gliotoxin (Glio), gliotoxin+CD14 (Glio+rCD14), gliotoxin+pMO2 (0.5 or 0.25 mg/ml equivalent to 0.2 and 0.1 mM), and gliotoxin+the scrambled peptide (Glio+pSCR). Cells grown in medium served as a control (none). Apoptosis is assayed by measuring DioC6(3) staining. Notably, recombinant CD14 and pMO2 (but not pSCR) equally protect lymphocytes from gliotoxin-induced apoptosis.

FIG. 12 is a graph showing the percentage of apoptotic cells in CD3+ cultures treated with gliotoxin (Glio), gliotoxin+CD14 (Glio+rCD14), gliotoxin+pMO2 (0.5 mg/ml), and gliotoxin+the scrambled peptide (Glio+pSCR). Cells grown in medium served as a control (none). Apoptosis is assayed by measuring 7AAD staining.

FIG. 13 is a graph showing the percentage of apoptotic cells in CD3+ cultures treated with gliotoxin (Glio), gliotoxin+CD14 (+rCD14), gliotoxin+pMO2 (0.5 or 0.25 mg/ml), and gliotoxin+the scrambled peptide (+pSCR). Cells grown in medium served as a control (none). Apoptosis is assayed by measuring cell size.

FIG. 14 is a graph showing the titration of pMO2 anti apoptotic activity. The graph summarizes four different experiments using 0.05, 0.1 and 0.2 mM pMO2. Numbers represent percent protection from apoptosis in the presence of pMO2 and gliotoxin as compared to gliotoxin and a control diluent.

FIG. 15 is a graph showing the titration of pMO2 anti apoptotic activity. The graph summarizes six different experiments using 0.1 and 0.2 mM pMO2, and 0.2 mM pSCR. Numbers represent percent protection from apoptosis in the presence of pMO2 or pSCR and gliotoxin as compared to gliotoxin and a control diluent.

FIG. 16 is a FACS histogram showing streptavidin-APC fluorescence of gated CD3 positive cells. Mononuclear cells were incubated for three hours with pMO2-biotin or DMSO (peptide diluent) as a control. Streptavidin-APC was measured with cell permeabilization (A) or without cell permeabilization (B).

FIG. 17 is a graph showing intracellular binding of pMO2-biotin. The graph demonstrates the percentage of cells showing pMO2 staining

as a function of time of incubation with pMO2-biotin, in CD3 positive and CD3 negative cells in vitro.

FIG. 18 is a graph showing dose response of MO2 induction in CD3+ lymphocytes by gliotoxin.

FIG. 19 is a graph representing the percentage of apoptotic lymphocytes and the percentage of MO2 positive lymphocytes in six different individuals (represented by separate bars), in control culture or in gliotoxin culture.

FIG. 20 is a dot plot of one experiment showing that MO2 induction and apoptosis induction occur in different cells. Apoptotic T cells (as measured by 7AAD) do not express MO2 and MO2 positive T cells are not apoptotic, after induction with gliotoxin. 20A no gliotoxin/staining with control antibody; 20B no gliotoxin/staining for MO2; 20C gliotoxin treatment/staining with control antibody; 20D gliotoxin treatment/staining for MO2.

FIG. 21 is a graph representing analysis of six different individuals (presented as a mean of 6 individuals) showing that apoptotic T cells (as measured by 7AAD) do not express MO2 and MO2 positive T cells are not apoptotic, after induction with gliotoxin.

### DETAILED DESCRIPTION OF EMBODIMENTS

### Definitions

As used herein the term “cell death” refers to a process by which a cell is terminally destroyed. Generally, cell death can occur through a mechanical injury, an assault by injurious agents (e.g. cytotoxic agents) termed “necrosis” or via a process of “programmed cell death” (also termed “apoptosis”).

Cell death can also occur via additional mechanisms such as “autophagy” defined as a process in which cell proteins and organelles are degraded by lysosomal proteases, and “mitotic catastrophe” characterized by aberrant mitotic spindle and formation of multiple micronuclei.

Cells dying by necrosis due to mechanical disruption or cytotoxic agents undergo a characteristic series of changes: the cells and their organelles, e.g. the mitochondria, swell (because the ability of the plasma membrane to control the passage of ions and water is disrupted); and the cell content leaks out, typically leading to inflammation of surrounding tissues.

In contrast, cells that die by apoptosis undergo a different series of changes: the cells shrink; they develop bubble-like blebs on their surface; the chromatin in their nucleus is degraded; the mitochondria break down leading to the release of cytochrome C; the cells break into small, membrane-wrapped, fragments; the phospholipids, phosphatidylserine, which is normally hidden within the plasma membrane, is exposed on the surface, leading to recognition by receptors on phagocytic cells (e.g. macrophages and dendritic cells) which then engulf the cell fragments and secrete cytokines that inhibit inflammation.

These characteristic changes often serve as a basis for designing assays to measure apoptosis in cell cultures as described more fully below.

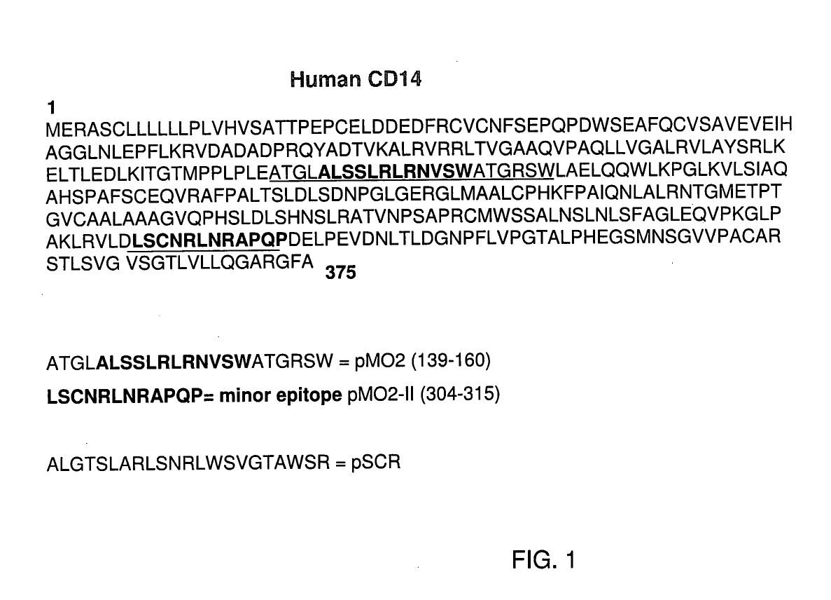

As used herein the term “CD14” also known as Cluster of Differentiation 14 encompasses the CD14 polypeptide. The present invention specifically refers to the soluble form of CD14 (“soluble CD14”) which is not anchored to the cell membrane. The native amino acid sequence of CD14 is presented herein as SEQ ID NO: 5, and is also presented in amino acids 1-375 of FIG. 1. “Native” CD14 refers to the CD14 polypeptide having the same amino acid sequence as a CD14 derived from nature.

As used herein the term “CD14 peptide” refers to peptides derived from the amino acid sequence of CD14, and variants thereof (which are further defined herein). The CD14 peptides may be isolated from a variety of sources, such as from human tissues or from another source, or prepared by recombinant or synthetic methods.

The terms “pMO2” and “pMO2II” refer to CD14 peptides bound by the anti CD14 antibody MO2. pMO2 is presented herein as SEQ ID NO: 1 and pMO2II is presented herein as SEQ ID NO: 3.

The term “variant” as used herein refers to a polypeptide or a peptide comprising a fragment, analogue, derivative, complex form, salt form or truncated version of a native CD14 or CD14 peptide, which retain some or all of the cell protective activity (preferably anti-apoptotic activity) of the corresponding native CD14 or CD14 peptide. The CD14 variant has at least about 80% amino acid sequence identity with the sequence of CD14 (SEQ ID NO: 5).

As a non-limiting example, the term pMO2 “variant” as used herein refers to a peptide comprising a fragment, analogue, derivative, complex form, salt form or truncated version of a full-length pMO2 peptide and which retains some or all of the cell protective activity (preferably anti-apoptotic activity) of the corresponding pMO2 peptide. The pMO2 variant has at least about 80% amino acid sequence identity with the sequence of pMO2 (SEQ ID NO: 1). Preferably, the amino acid sequence identity is at least about 85%, more preferably at least about 90%, and even more preferably at least about 95%. pMO2 variants include, for instance, pMO2 peptides wherein one or more amino acid residues are added, or deleted, at the N- or C-terminus of SEQ ID NO: 1. pMO2 variants also include pMO2 fragments, which retain the cell protective activity (preferably anti-apoptotic activity) of the complete pMO2 peptide. In one embodiment such pMO2 fragments are at least 15 amino acids long. Variants also comprise pMO2 peptides in which one or more of the amino acids were substituted or otherwise modified, but which retain the cell protective (preferably anti-apoptotic activity) of pMO2.

“Percent (%) amino acid sequence identity” with respect to sequences identified herein is defined as the percentage of amino acid residues in a candidate sequence that are identical with the amino acid residues in the sequences herein defined as SEQ ID NOS 1-5, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

“Protective activity” in the context of CD14 or CD14 peptides of the invention refers to the ability of such molecules to protect cells from cell death. A preferred cell protective activity is an anti apoptotic activity.

An “isolated” CD14 peptide is a peptide having an amino acid sequence that is derived from the CD14 polypeptide, but that is separate and/or recovered from the CD14 polypeptide, or that has been newly synthesized, and which does not ordinarily exist in the isolated form in nature.

The term “antibody” is used in the broadest sense and specifically covers, without limitation, monoclonal antibodies and polyclonal antibodies. The term “monoclonal antibody” as used herein refers to an antibody obtained from a population of substantially homogenous antibodies, i.e. the individual antibodies comprising the population are identical except for possible naturally-occurring mutations that may be present in minor amounts.

The term “medical condition associated with apoptotic cell death” means a disease or clinical event in which apoptosis is part of the pathology of the disease. The “pathology” of a disease includes all phenoma that compromise the well-being of the patient. Examples of such medical conditions include without limitation, stem cell transplantation (including hematopoietic stem cell transplantation), immune deficiencies, infectious diseases (including for example, HIV), autoimmune diseases, central nervous system (CNS) disorders such as neurodegenerative diseases, Alzheimer's disease (AD), Parkinson's disease (PD) and amyotrophic lateral sclerosis (ALS), and in the more acute conditions of cerebral ischemia, traumatic brain injury (TBD, and spinal cord injury (SCI), heart diseases, aging, and cancer including cell damage and apoptotic death induced by chemotherapy side effects, for example apoptosis of gastrointestinal epithelial cells. Examples of autoimmune diseases include, without limitation, inflammatory bowel disease, systemic lupus erythematosus, rheumatoid arthritis, juvenile chronic arthritis, spondyloarthropathies, systemic sclerosis, (scleroderma), multiple sclerosis, diabetes.

“Tumor” as used herein refers to all neoplastic cell growth and proliferation whether malignant or benign, and all pre-cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma and leukemia. More particular examples of such cancers include breast cancer, prostate cancer, colon cancer, squamous cell cancer, small-cell lung cancer, non-small-cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, liver cancer, vulval cancer, thyroid cancer, hepatic carcinoma and various types of head and neck cancer.

The term “mammal” as used herein refers to any animal classified as a mammal, including, without limitation, humans, domestic and farm animals, and zoo, sports or pet animals such as horses, pigs, cattle, dogs, cats, ferrets etc. In a preferred embodiment of the invention, the mammal is a human.

As used herein, the term a “therapeutically effective” amount is an amount which prevents or delays the onset or progression of an indicated disease or other adverse medical condition. The term also includes an amount sufficient to arrest or reduce the severity of an ongoing disease or other adverse medical condition, and also includes an amount necessary to enhance normal physiological functioning.

As used herein, “treatment” of a disease or other adverse medical condition, should be broadly interpreted as variously including palliative, active, causal, conservative, medical, prophylactic, and/or symptomatic treatment, treatment designed to delay the onset or progression of the disease or other adverse medical condition, as well as treatment designed to arrest or reduce the severity of an ongoing disease or other adverse medical condition.

As used herein, a “pharmaceutically acceptable” component (such as a salt, carrier, excipient or diluent) of a composition according to the present invention is a component which (1) is compatible with the other ingredients of the composition in that it can be combined with CD14 or the anti apoptotic peptides of the invention without eliminating the biological activity of CD14 or the peptides; and (2) is suitable for use in non-human mammals or humans without undue adverse side effects (e.g., toxicity, irritation, and allergic response). Side effects are “undue” when their risk outweighs the benefit provided by the pharmaceutical composition.

Administration “in combination with” one or more further therapeutic agents includes simultaneous (concurrent) and consecutive administration in any order.

The terms “administer” “administration” and the like, as used herein with reference to CD14 or a peptide, are intended to be inclusive of any means for delivering the polypeptide or the peptide to a subject.

The term “mononuclear cells” refers to both lymphocytes and monocytes. The term “lymphocyte” denotes a type of white blood cell in the vertebrate immune system. Lymphocytes include natural killer cells (NK cells), T cells and B cells.

### Compositions and Methods of the Invention

The present invention is based on the finding that administration of recombinant human soluble CD14 blocks apoptosis of human lymphocytes in vitro. Moreover, a synthetic peptide corresponding to the MO2 epitope of CD14 was found to fully retain the apoptosis protective effect of the CD14 protein.

Surprisingly, the MO2 peptide was found to penetrate lymphocytes in vitro when administered to the culture medium.

Without wishing to be bound by theory, the inventors propose that soluble CD14 may bind under certain circumstances to lymphocytes, possibly but not necessarily via membrane structures which may be differentially expressed or temporarily expressed by various lymphocyte populations (15), and is rapidly internalized. The internalized molecule and specifically the MO2 epitope inhibit apoptotic processes inside the cell. As such CD14 or fragments thereof, specifically the MO2 peptide, may serve as a “molecular bandage” and may be applied as a therapeutic composition for treatment of diseases associated with apoptotic cell death.

### Assays of Apoptosis

Assays of apoptosis are designed to measure cell viability or cellular destruction that occurs as a result of the apoptotic process. Various methods for assessing apoptosis are known in the art. The following are several non-limiting examples of such methods.

### 1. Measurement of Mitochondria Membrane Potential:

During apoptosis, the mitochondria membrane potential is considerably reduced and eventually lost. The reduction is characteristics of early apoptosis whereas complete loss occurs upon cell death.

The changes in the mitochondria membrane potential can be measured with 3,3′-Dihexyloxacarbocyanine iodide (Dioc6(3)). The binding to the mitochondria membrane is relative to its potential, the highest binding occurs in live and intact cells; the lowest in dead cells. The binding is monitored by flow cytometry (FL1 channel).

After overnight culture (see below), cells are washed in PBS and are treated with 40 nM DiOC6(3) for 15 min at 37° C. Cells are then washed in PBS, stained for lymphocytes surface markers such as CD3/CD4/CD8, with the appropriate antibodies, for 30 min at 4° C., washed again in PBS, resuspended in 0.5 ml PBS and analyzed using the FACS (flow cytometry).

2. Measurement of 7-amino-actinomycin D (7AAD) Nuclei Staining:

Cells in the later stages of apoptosis, and dead cells, have lost plasma membrane integrity and are permeable for 7AAD.

7AAD is a nucleic acid dye that binds to single stranded DNA, selectively to GC regions of DNA. No binding is observed in live cells. The binding to apoptotic cells is proportional to the degree of apoptosis and membrane damage. The 7AAD fluorescence is detected in the far-red range of the spectrum (650 nm), in the FL3 channel.

After overnight culture (see below), cells are washed with PBS and are stained with monoclonal antibodies (CD3/4/8) for 30 min at 4° C., then washed again in PBS. Cells are then incubated with 7AAD (20 μg/ml) for 20 min at 4° C. in the dark and directly analyzed using the FACS (flow cytometry).

### 3. Measurement of Cell Size:

As a result of the process of apoptosis, cells undergo a cytoplasm shrinkage, which can be detected as a decrease in cell size as observed by the forward scatter position of the cells analyzed by FACS; the smaller lymphocytes are apoptotic, the larger are intact.

### Peptides

The present invention provides a newly identified peptide which exhibits cell protective activity against cell death, specifically apoptosis. The peptide of the invention was identified as follows: A random (5460) 12-mer peptide mapping of human CD14 using the MO2 antibody (monoclonal anti-human CD14) was performed as follows:

The solid-phase bound peptides were screened through credit-card format mini-PEPSCAN cards (455 peptides/card) as described previously (17; WO 93/09872). All peptides were acetylated at the amino terminus. The binding of antibodies to the peptides was tested in a PEPSCAN-based enzyme-linked immuno assay (ELISA). The 455-well credit-card-format polypropylene cards, containing the covalently linked peptides, were incubated with antibody dissolved in a PBS-based blocking-buffer which contains 5% horse-serum (v/v) and 5% ovalbumin (w/v)) and 1% Tween-80 (4° C. overnight). After washing the peptides with PBS (pH 7.4) the peptides were incubated with e.g. anti-mouse antibody peroxidase (dilution 1/1000) (one hour, 25° C.), and subsequently, after washing with PBS (pH 7.4), with the peroxidase substrate 2,2′-azino-di-3-ethylbenzthiazoline sulfonate (ABTS) and 2 μl/ml 3% H2O2. After one hour, the colour development is measured. The colour development of the ELISA is quantified with a CCD-camera and an image processing system. The quantification set-up consists of a CCD-camera, an Image Processing Software and a Pentium computer system.

The mapping procedure resulted in the identification of the major MO2 epitope delineated between amino acids 143-154 of the human CD14 sequence, consisting of the amino acids: ALSSLRLRNVSW (SEQ ID NO: 2). An additional epitope, consisting of the amino acids LSCNRLNRAPQP (SEQ ID NO: 3)—a minor one (based on the signal strength obtained in the ELISA) has also been characterized by the same methodology.

A peptide encoding amino acids 139-160 (22 amino acids: ATGLALSSLRLRNVSWATGRSW; SEQ ID NO: 1) of the human CD14 was synthesized and termed pMO2. A scrambled peptide based on the same amino acids was also synthesized and named pSCR (ALGTSLARLSNRLWSVGTAWSR; SEQ ID NO: 4). Amino acid sequence of CD14 (SEQ ID NO 5), as well as the sequence of the various peptides is shown in FIG. 1. Both the pMO2 and pSCR peptides have a molecular weight of 2630 kDa. The peptides were labeled with biotin at the N-terminal.

The peptides may be prepared by any method known in the art, preferably by synthetic means.

The present invention also contemplates a fragment of CD14 which retains the anti apoptotic activity of the complete molecule. An example of a CD14 fragment that can be used in accordance with the invention is a CD14 fragment which is devoid of the LPS binding domain. In such a fragment the anti-apoptotic activity is retained while the activities which are mediated by LPS and may have detrimental consequences are abolished. The crystal structure of CD14 demonstrating the LPS binding domain can be viewed in Kim et al [19].

### CD14 and CD14 Peptide Variants

In addition to the full length CD14 peptide (e.g. pMO2) sequences described herein, it is contemplated that peptide variants can be prepared. CD14 peptide variants can be prepared by introducing appropriate amino acid changes during the synthesis of the peptides.

Variations in CD14 may be achieved by introducing variations in the native full-length nucleic acid sequence of CD14. Such variations can be made, for example, using any of the techniques and guidelines for conservative and non-conservative mutations set forth, for instance in U.S. Pat. No. 5,364,934. Variations may be a substitution, deletion or insertion of one or more codons encoding the CD14 polypeptide that results in a change in the amino acid sequence of CD14 as compared with the native sequence.

Optionally the variation is by substitution of at least one amino acid with any other amino acid in the CD14 molecule or CD14 peptide. Guidance in determining which amino acid residues may be inserted, substituted or deleted without adversely affecting the cell protective activity may be found for example by comparing the sequence of human CD14 with that of homologous known proteins and minimizing the number of amino acid sequence changes made in regions of high homology. Amino acid substitutions can be the result of replacing one amino acid with another amino acid having similar structural and/or chemical properties, such as the replacement of a leucine with a serine, i.e. conservative amino acid replacements. Insertions or deletions may optionally be in the range of 1 to 5 amino acids. The variation allowed may be determined by systematically making insertions, deletions or substitutions of amino acids in the sequence and testing the resulting variants for cell protective activity, as indicated, for example in the apoptosis assays described above and in the Examples below.

The variations can be made using methods known in the art.

For example, for the CD14 peptides the variations can be made by de novo synthesis of peptides according to an altered amino acid sequence.

For the complete CD14 molecule, such methods include site-directed mutagenesis, cassette mutagenesis, restriction selection mutagenesis or other known techniques that can be performed on the cloned DNA to produce the CD14 variant DNA.

Additional modifications resulting in stabilization of the peptides are also contemplated by the present invention. Such modifications include for example, blocking the N or C terminal, and cyclization.

### EXAMPLES

### Cell Culture

Mononuclear cells, freshly obtained from peripheral blood on a Ficoll-Hypaque gradient are washed in PBS and resuspended in RPMI supplemented with 10% fetal calf serum, glutamine, antibiotics, non-essential amino acids and sodium pyruvate, at a concentration of 4×10⁶ cells per ml. Cells (0.5 ml per well) are incubated overnight, in 24 well plates, in the presence of Gliotoxin (0.1 μg/ml) or PMA (1 μg/ml) and with pMO2 or pSCR peptides (0.2, 0.1, 0.05 mM equivalent to 0.5, 0.25 and 0.125 mg/ml); as a control DMSO—the diluent of the peptides—was used at the appropriate dilution or with recombinant human CD14 (10 and 5 μg/ml). For the experiments aimed at neutralizing the human rCD14, monoclonal antibodies against CD14 or against actin (as a control) are added, together with the CD14, at a final concentration of 15 μg/ml.

After the overnight incubation, cells are processed for apoptosis assessment using the methods described above.

### Example 1

### Soluble CD14 Protects Lymphocytes from Induced Apoptosis In Vitro

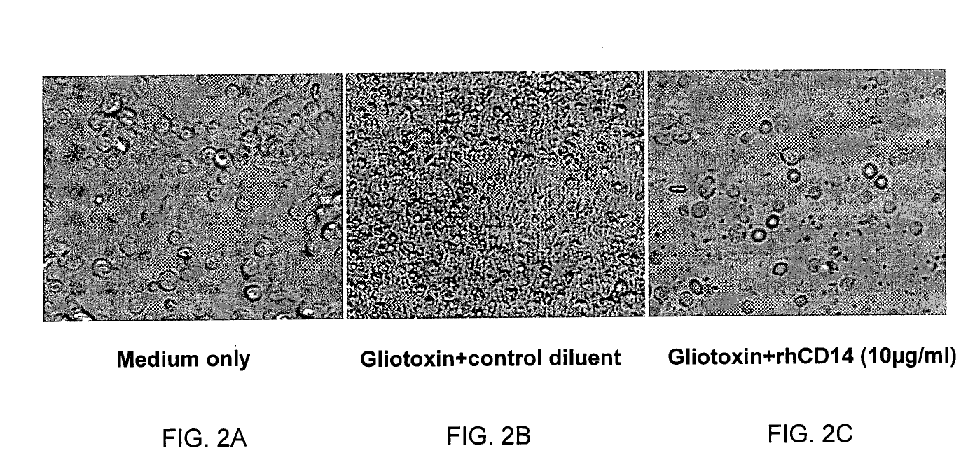

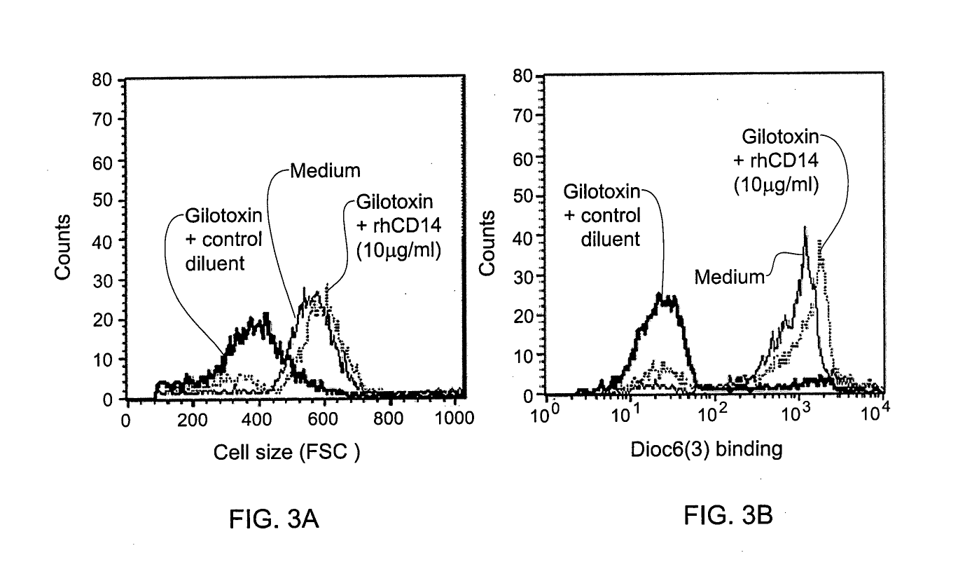

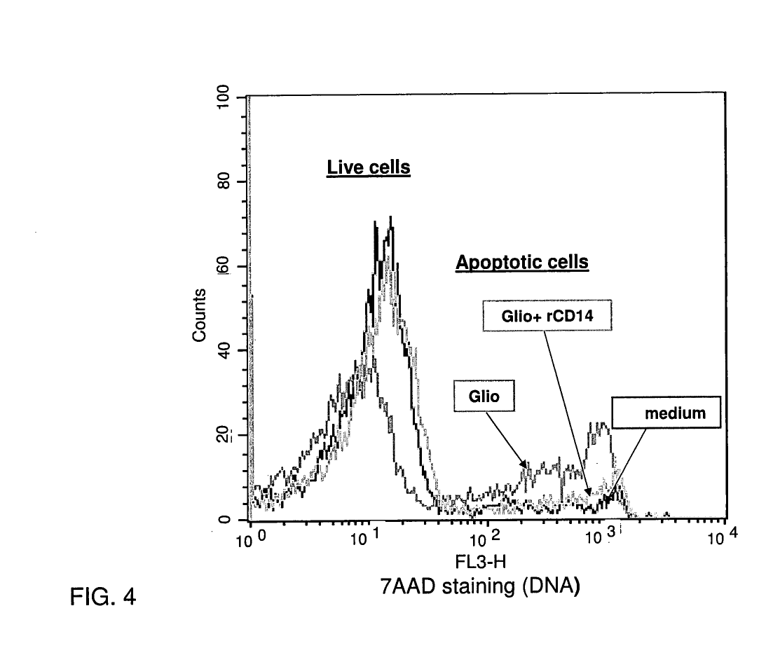

The effect of recombinant human CD14 on the induction of apoptosis in human lymphocytes and in human CD3 positive lymphocytes was tested in vitro. Human mononuclear cells obtained from healthy individuals were incubated with apoptosis inducers, gliotoxin or PMA. Addition of human recombinant CD14 during the incubation blocked significantly the apoptosis of lymphocytes and of CD3 positive lymphocytes. Namely, recombinant human CD14 protects lymphocytes from gliotoxin-induced apoptosis. As can be seen in FIG. 2, the size of the cells is decreased by exposure to gliotoxin while treatment with rhCD14 restores the size of the majority of cells to normal. FIG. 3 demonstrates FACS analysis of the CD3 positive cells shown in FIG. 2. Two parameters were measured: cell size (A), and mitochondrial membrane potential determined by Dioc6 (3) binding (B). In (A) there is a shift to the left (characteristic to apoptotic cells) under gliotoxin treatment compared to “medium”, and a reversal to normal size in the presence of rhCD14. In (B) low binding of Dioc6 (3) in the presence of gliotoxin (characteristic to apoptotic cells) can be observed, and the effect is reversed by rhCD14. This is also demonstrated in FIG. 4 using 7AAD staining.

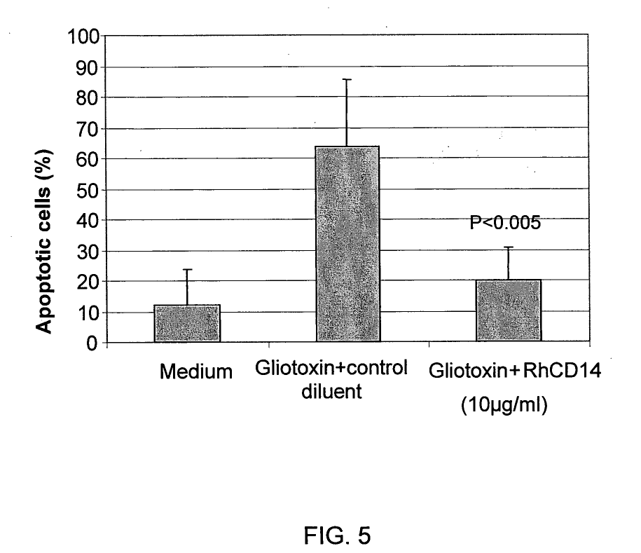

FIG. 5 summarizes a number of experiments conducted with the DiOC6 (3) staining and also demonstrates the statistical significance of the CD14 blocking effect.

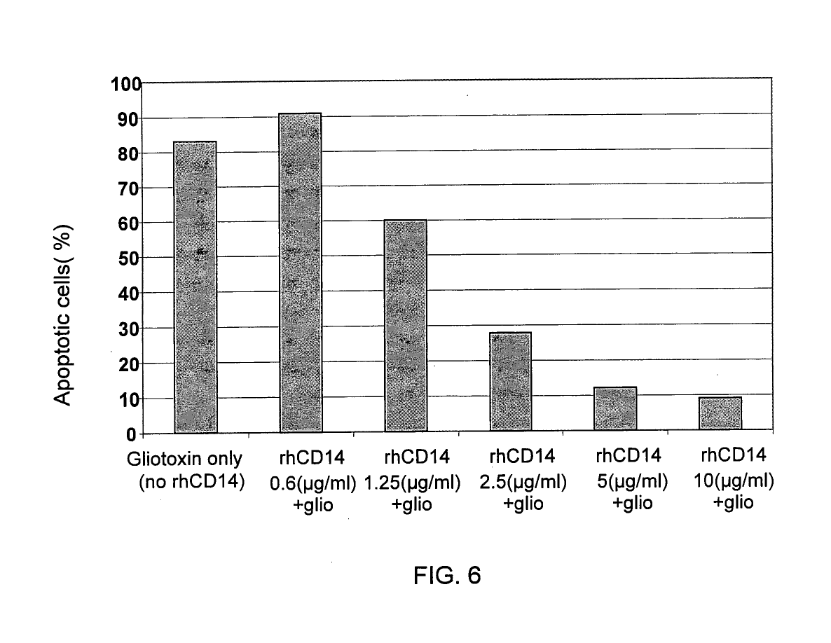

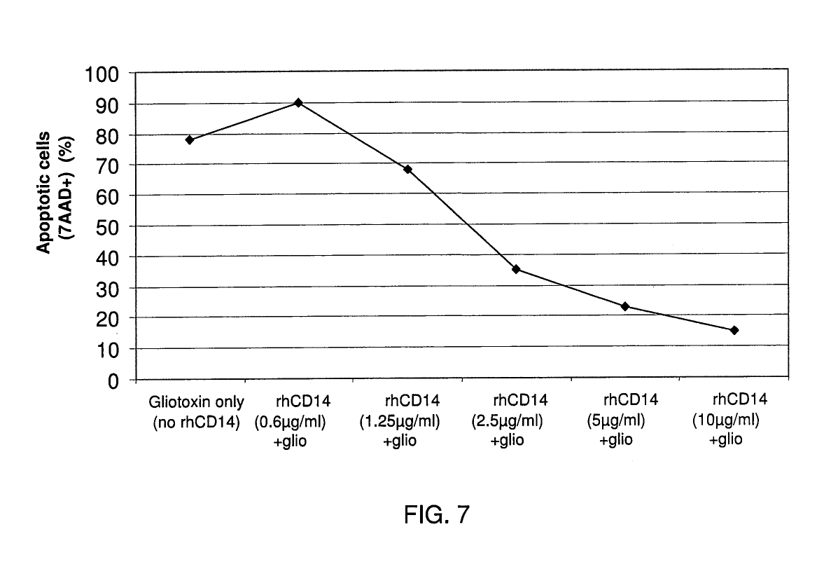

In addition, in order to investigate the spectrum of effective concentrations of rhCD14, mononuclear cells were cultured overnight in the presence of gliotoxin and varying concentrations of rhCD14, ranging from 10 to 0.6 μg/ml. Apoptosis of CD3+ cells was measured with Dioc6(3). As expected, cells cultured without gliotoxin included a very low number of apoptotic cells, about 0.3% of the cultured cells. As shown in FIG. 6 rhCD14 was effective in inhibiting apoptosis already at a concentration of 1.25 μg/ml, demonstrating a clear dose response correlating the anti apoptotic effect with increasing concentrations of rhCD14. Apoptosis was also measured by 7AAD binding, as shown in FIG. 7, demonstrating similar results.

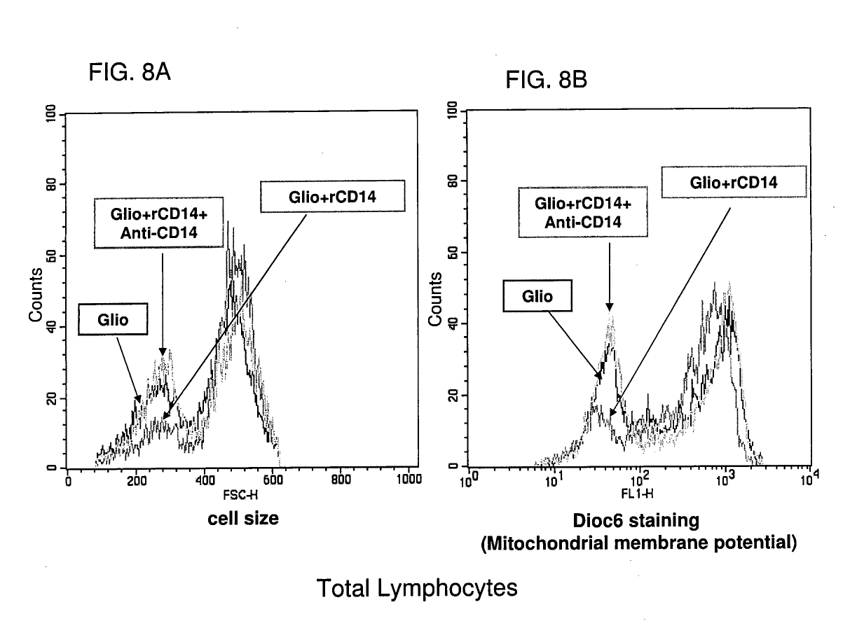

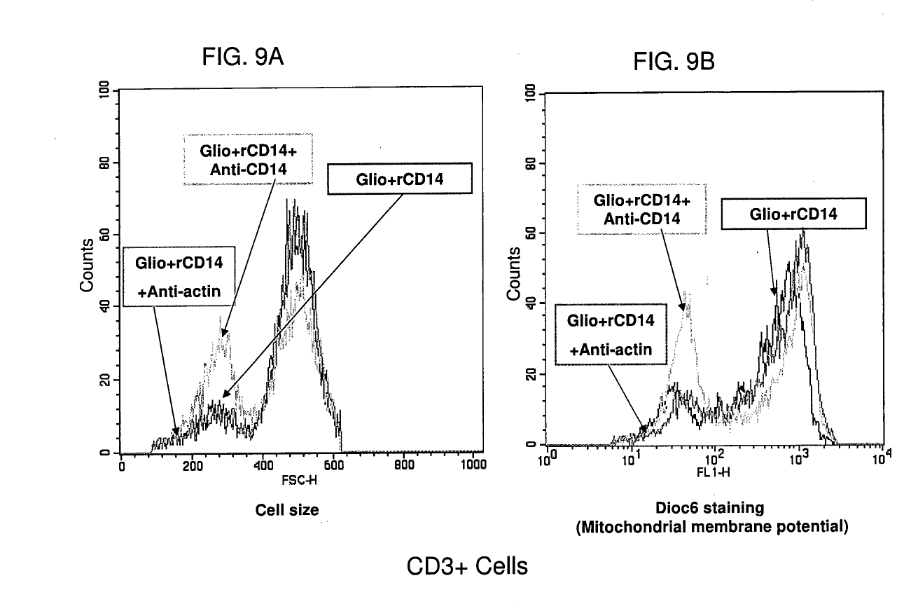

This blocking effect was completely neutralized with anti-CD14 monoclonal antibodies but not with a control irrelevant antibody (anti-actin). This is demonstrated in FIGS. 8 and 9 for all lymphocytes or CD3 positive lymphocytes as measured by cell size or by DiOC6 (3) staining.

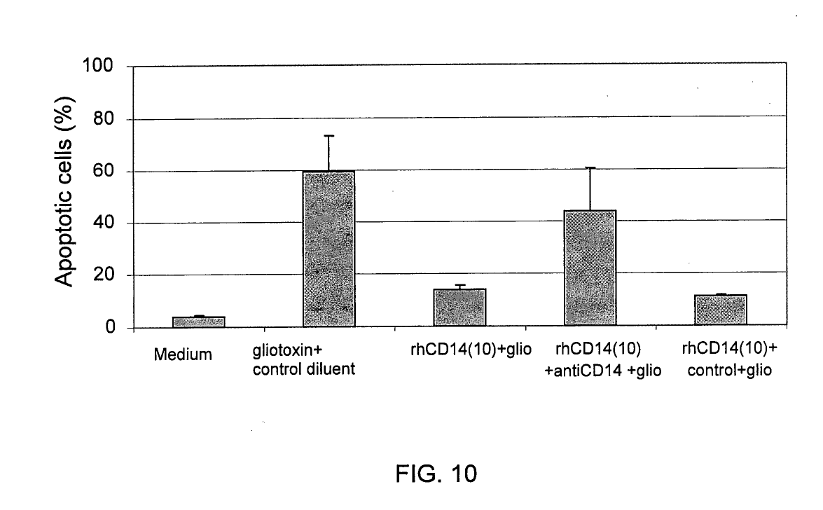

FIG. 10 shows a summary of three different experiments, testing neutralization of the anti apoptotic effect of rhCD14 (10 μg/ml) by monoclonal anti CD14 antibodies (15 μg/ml). As a control, monoclonal anti actin antibodies of same isotype and concentration were used. As can be seen in FIG. 10, the effect of rhCD14 is neutralized by anti-CD14 antibodies, while the control antibodies were ineffective and did not neutralize the CD14 anti apoptotic effect

### Example 2

### pMO2 Protects Lymphocytes from Induced Apoptosis In Vitro

The inventor of the present invention has recently described a novel lymphocyte subpopulation of T cells expressing an intracellular CD14-like antigen, designated MO2. MO2 is a commercial monoclonal antibody directed against human CD14 (14, 18).

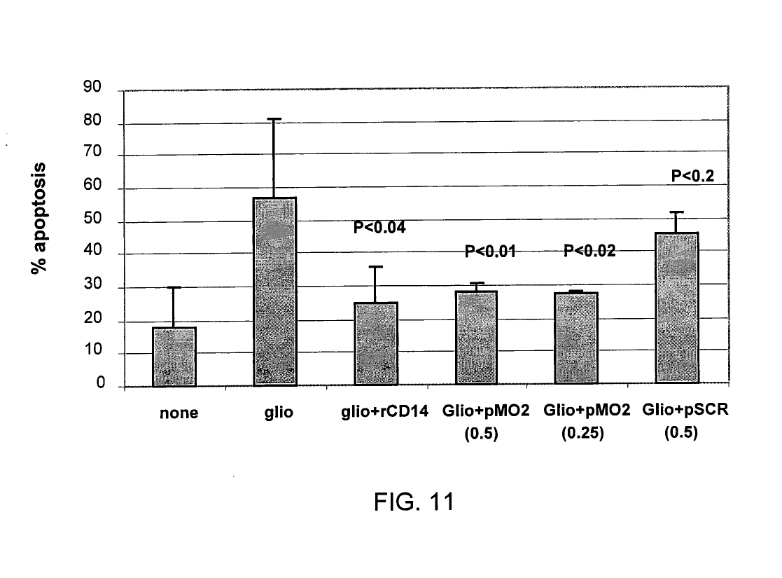

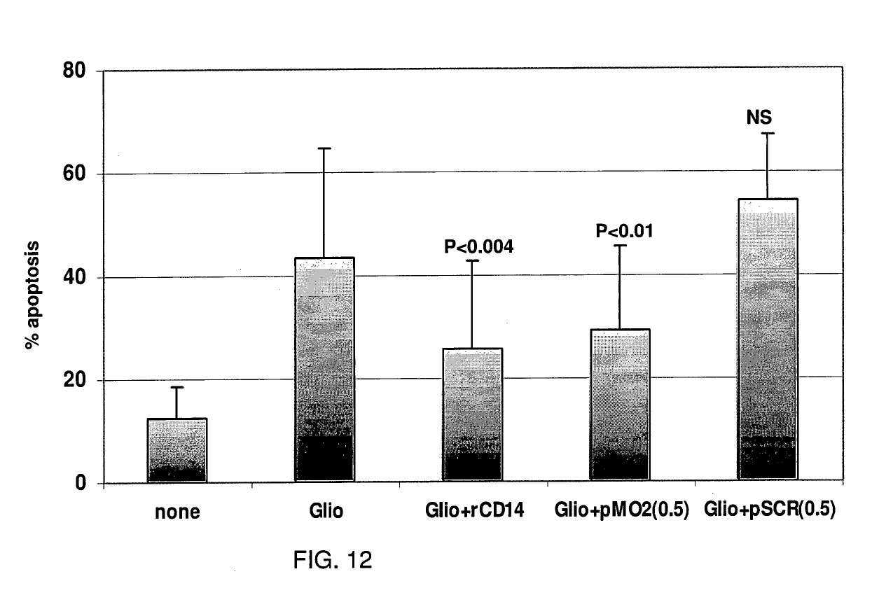

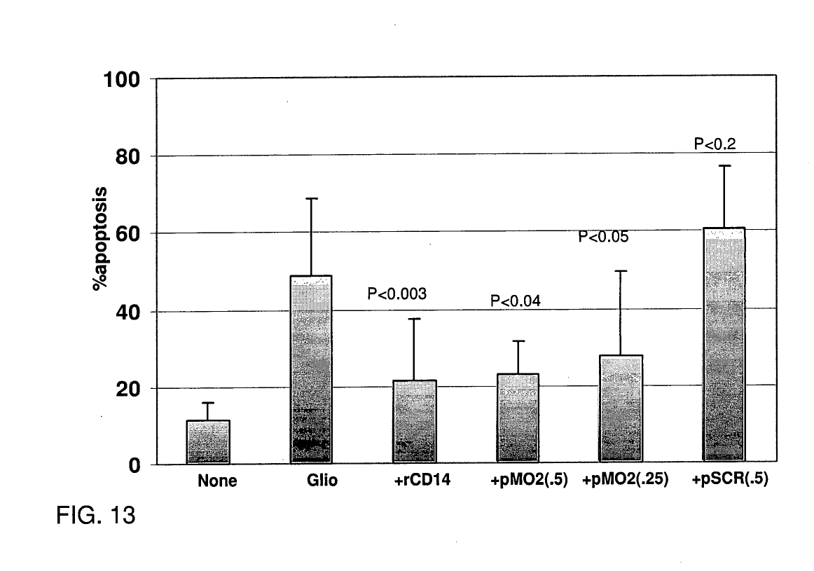

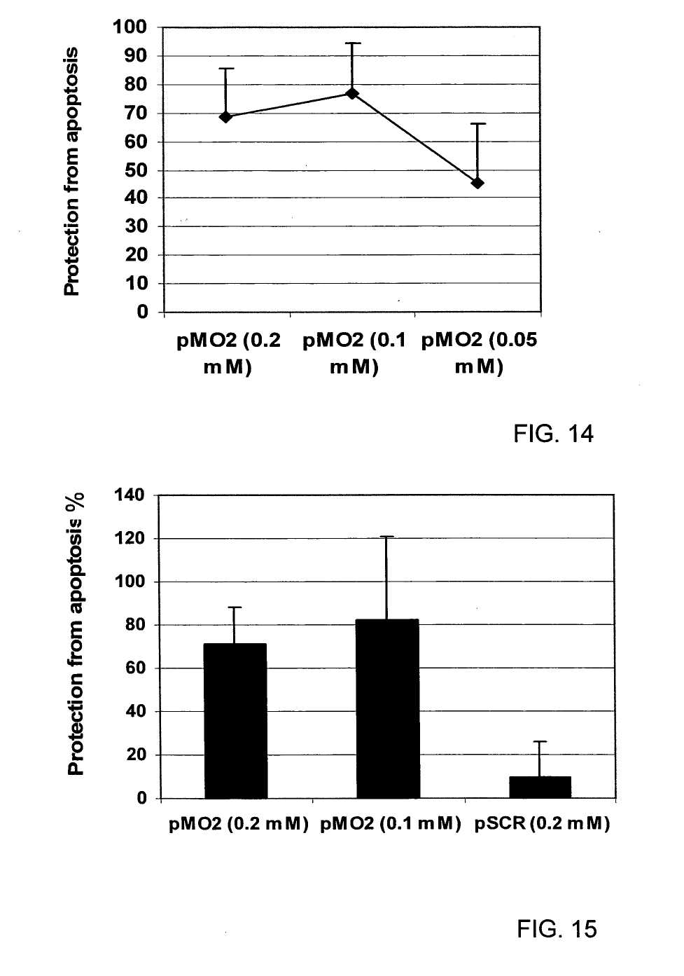

As explained above, the epitopes recognized by this monoclonal MO2 antibody were mapped using a random peptide library, and one of the specific peptides (amino acids 139-160 of CD14; pMO2) was synthesized. The MO2 peptide (pMO2) was used in the apoptosis inducing system detailed above, and was found to retain the anti-apoptotic effect of the whole recombinant human CD14. A scrambled peptide (pSCR) composed of the same amino acids but in a scrambled sequence, was used as a control. The scrambled peptide did not show any protective effect in vitro. This is shown in FIG. 11 for DiOC6 (3) staining, in FIG. 12 for 7AAD staining and in FIG. 13 for cell size. FIG. 14 shows a dose response curve of the pMO2 anti apoptotic activity. 0.1 mM and 0.2 mM pMO2 showed a very high efficiency in protecting cells from apoptosis and even 0.05 mM pMO2 provided about 45% protection. As can be seen in FIG. 15, pSCR, the scrambled peptide provided no protection at 0.2 mM. Percent protection is calculated as follows: 1−(% apoptosis in the presence of gliotoxin and CD14 minus background % apoptosis in medium only divided by % apoptosis in the presence of gliotoxin and control minus background % apoptosis in medium only)×100.

### Example 3

### Intracellular Binding of pMO2 to CD3 Positive Cells

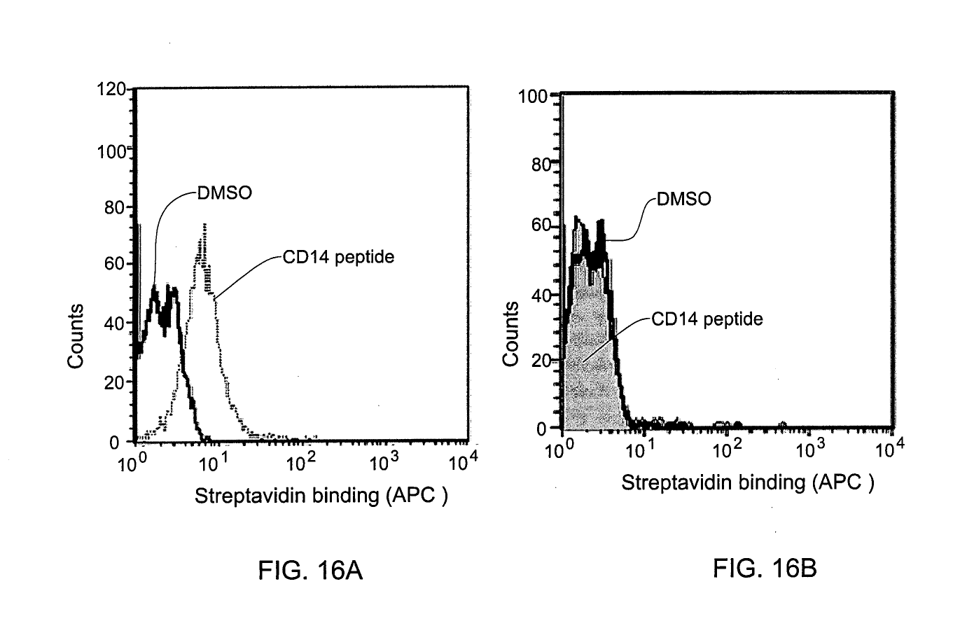

Biotin-labeled pMO2 was shown to enter mononuclear cells in vitro. The cells were incubated for 3 hours with pMO2-biotin or DMSO (the peptide diluent) as a control. In order to visualize the biotin-labeled pMO2, the cells were washed and stained with anti-CD3 antibodies and fluorescent streptavidin (streptavidin-APC), with or without prior fixation and permeabilization. Interestingly, as shown in FIG. 16, the pMO2 could be visualized only upon fixation and permeabilization of the cells (FIG. 16A). As can be seen in FIG. 16B, staining of lymphocytes with fluorescent streptavidin, as detailed above, without prior fixation and permeabilization did not result in any positive signal. These results clearly show that pMO2 penetrates lymphocytes and can be visualized within the cells, but not on their surface.

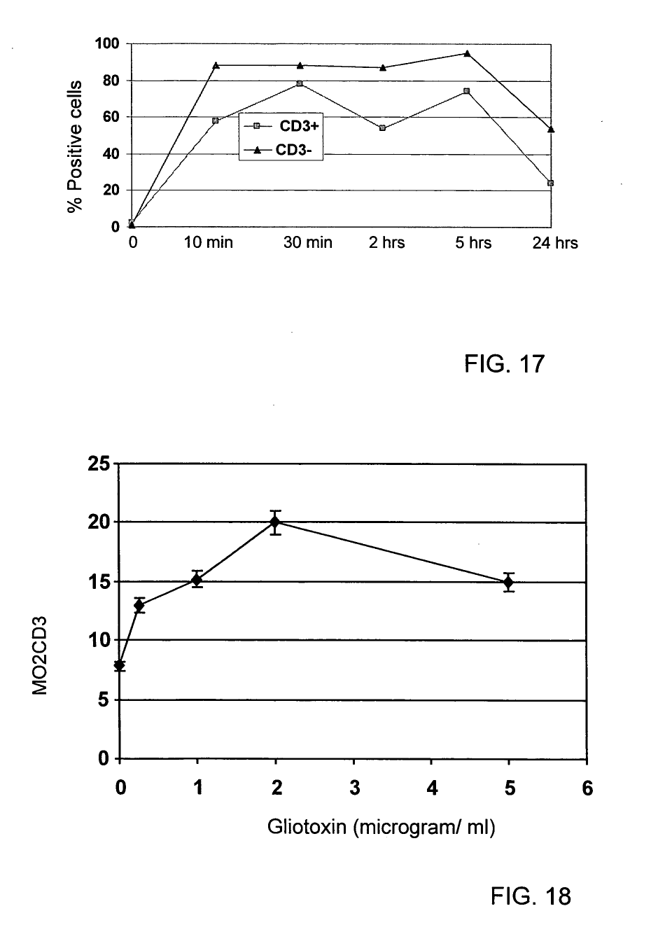

FIG. 17 demonstrates the kinetics of pMO2 penetration into CD3 positive and CD3 negative cells in vitro, and shows clearly that pMO2 penetrates the majority of the lymphocytes within 10 minutes of incubation.

Further support for the concept that lymphocytes are capable of MO2 uptake is found in another experiment in which monocyte secretion of CD14 in vitro was blocked with Brefeldin A (a drug blocking the Golgi system thus inhibiting cell secretion). Blocking cell secretion resulted in a substantial decrease in the intracellular expression of MO2 in lymphocytes, as evident by staining with the anti MO2 antibodies.

### Example 4

### Exposure to Apoptotic Agents Induces pMO2 Expression Only in Cells that Become Apoptosis Resistant

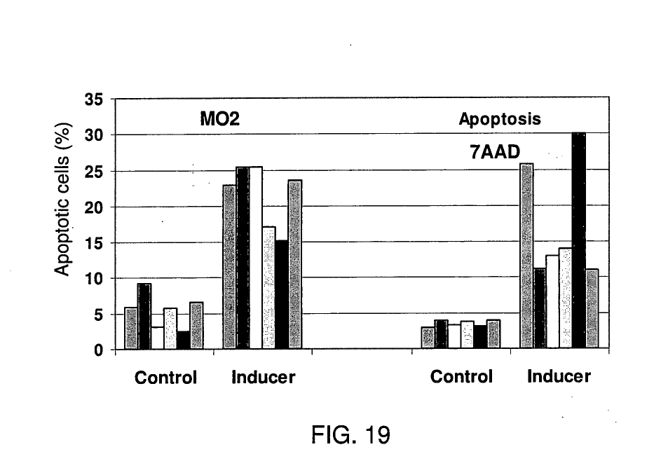

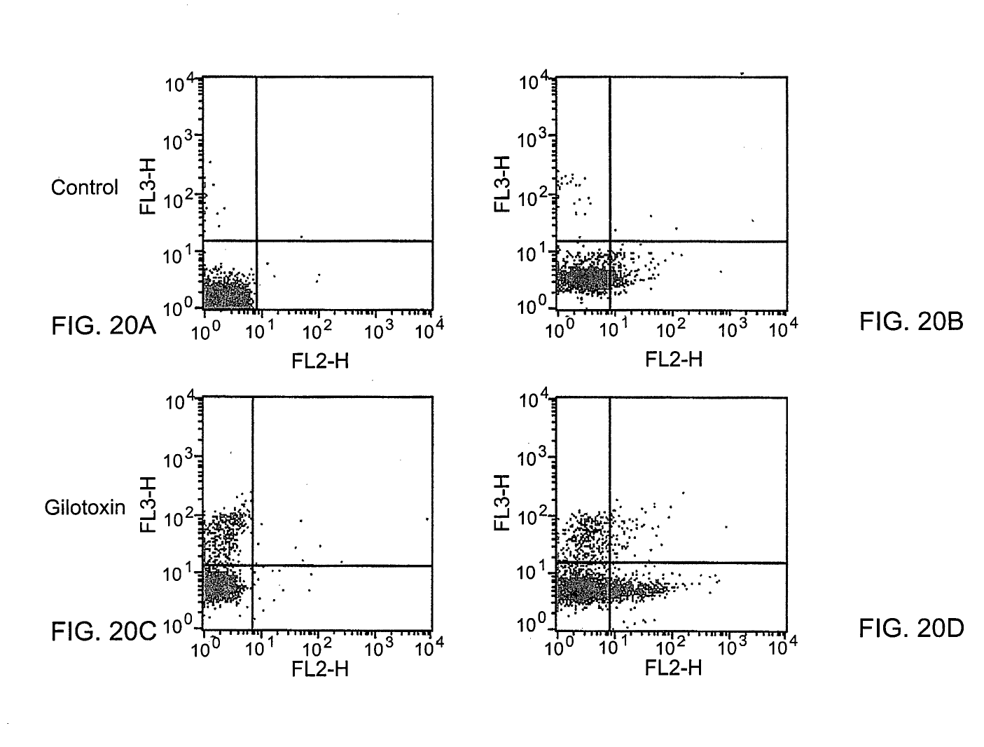

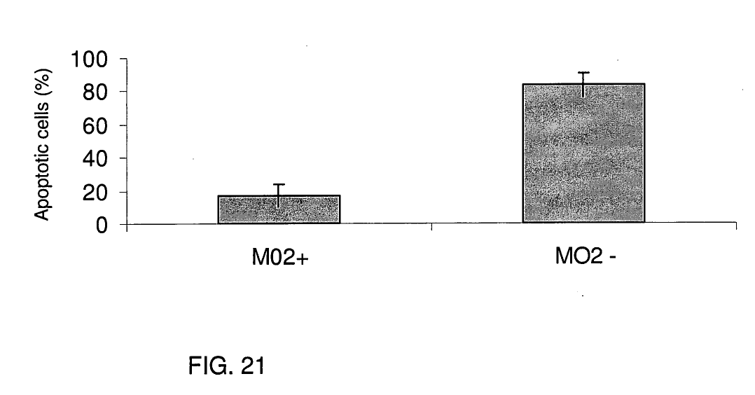

Mononuclear cells were cultured overnight in the presence of gliotoxin, fixed and permeabilized as described in Tartakovsky et al above, and were stained with the MO2 antibody, with 7AAD and with anti-CD3. The results showed that induction of apoptosis in lymphocytes was accompanied by the induction of MO2 (as shown in FIG. 18, dose response of gliotoxin). FIG. 19 shows the concomitant measurement of MO2 and of apoptosis (by 7AAD) in lymphocytes obtained from six different individuals. Interestingly, the MO2 antigen was detected particularly in the CD3 lymphocytes that were not induced to become apoptotic (FIG. 20). Indeed, examination of the apoptotic CD3 positive cells (as indicated by 7AAD staining), indicated that 82.9% of the cells were MO2 negative and only 17.1% of the cells were MO2 positive (FIG. 21); examination of the MO2 positive CD3 cells, revealed that 80.2% of the cells were non apoptotic (based on 7AAD staining) whereas only 19.8% of the cells were apoptotic. These results demonstrate that MO2 expression inside the CD3 positive cells is associated with a non-apoptotic status.

### REFERENCES

[1] A. Haziot, S. Chen, E. Ferrero, M. G. Low, R. Silber, S. M. Goyert, J. Immunol. 141 (1988) 547-552.
[2] R. R. Schumann, S. R. Leong, G. W. Flaggs, P. W. Gray, S. D. Wright, J. C. Mathison, P. S. Tobias, R. J. Ulevitch, Science 249 (1990) 1429-1431.
[3] D. L. Simmons, S. Tan, D. G. Tenen, A. Nicholson-Weller, B. Seed, Blood 73 (1989) 284-289.
[4] J. J. Durieux, N. Vita, O. Popescu, F. Guette, J. Calzada-Wack, R. Munker, R. E. Schmidt, J. Lupker, P. Ferrara, H. W. Ziegler-Heitbrock, Eur. J. Immunol. 24 (1994) 2006-2012.
[5] E. A. Frey, D. S. Miller, T. G. Jahr, A. Sundan, V. Bazil, T. Espevik, B. B. Finlay, S. D. Wright, J. Exp. Med. 176 (1992) 1665-1671.
[6]H. P A. Jersmann, Immunol. And Cell Biol. 83 (2005) 462-467.
[7] A. Devitt, O. D. Moffatt, C. Raykundalia, J. D. Capra, D. L. Simmons, C. D. Gregory, Nature 392 (1998) 505-509.
[8] V. A. Fadok, D. R. Voelker, P. A. Campbell, J. J. Cohen, D. L. Bratton, P. M. Henson, J. Immunol. 148 (1992) 2207-2213.
[9] S. Heidenreich, M. Schmidt, C. August, P. Cullen, A. Rademaekers, H. G. Pauels, J. Immunol. 159 (1997) 3178-3188.
[10] Y. Kanatani, T. Kasukabe, J. Okabe-Kado, Y. Yamamoto-Yamaguchi, N. Nagata, K. Motoyoshi, Y. Honma, Cell Growth Differ. 10 (1999) 705-712.
[11] Z. G. Fridlender, R. Rabinowitz, M. Schlesinger, Hum. Immunol. 60 (1999) 1028-1038.
[12] J. E. Rey Nores, A. Bensussan, N. Vita, F. Stelter, M. A. Arias, M. Jones, S. Lefort, L. K. Borysiewicz, P. Ferrara, M. O. Labeta, Eur. J. Immunol. 29 (1999) 265-276.
[13] K. H. Lue, R. P. Lauener, R. J. Winchester, R. S. Geha, D. Vercelli, J. Immunol. 147 (1991) 1134-1138.
[14] B. Tartakovsky, M. Fried, M. Bleiberg, D. Turner, M. Hoffman, I. Yust. Immunol Lett. 85 (2003) 35-40.
[15] N. Vita, S. Lefort, P. Sozzani, R. Reeb, S. Richards, L. K. Borysiewicz, P. Ferrara, M. O. Labeta, J. Immunol. 158 (1997) 3457-3462.
[16] Yu L C, Turner J R, Buret A G Exp. Cell Res. 312 (17): 3276-86 (2006)
[17] Slootstra-J W; Puijk-W C; Ligtvoet-G J; Langeveld-J P; Meloen-R H Mol-Divers. 1: 87-96 (1996)
[18] Turner et al, CDLI 11, 1040-1044, 2004
[19] Kim et al., J. Biol. Chem. Vol. 280, No. 12 pp 11347-11351, 2005

1.-51. (canceled)

52. An isolated CD14 peptide which exhibits protective activity against cell death.

53. The isolated CD14 peptide of claim 53, wherein the CD14 peptide comprises SEQ ID NO. 1, or SEQ ID NO. 3, or any variant thereof.

54. A pharmaceutical composition for protecting cells against cell death, comprising: as an active ingredient a polypeptide having at least 80 percent amino acid sequence identity to the amino acid sequence of a soluble CD14 polypeptide (SEQ ID NO: 5), a fragment thereof, or at least one CD14 peptide derived therefrom which retain a protective activity of the complete soluble CD14 polypeptide, and a pharmaceutically acceptable carrier.

55. The pharmaceutical composition of claim 54, wherein the at least one CD14 peptide comprises an amino acid sequence of SEQ ID NO: 1, SEQ ID NO: 3 or any variant thereof.

56. The pharmaceutical composition of claim 54, wherein the pharmaceutical composition comprises a combination of at least two CD14 peptides.

57. The pharmaceutical composition of claim 56, wherein the at least two CD14 peptides comprise an amino acid sequence of SEQ ID NO: 1 and SEQ ID NO: 3 or any variants thereof.

58. The pharmaceutical composition of claim 54, wherein the cell death is caused by apoptosis and wherein the protective activity is an anti-apoptotic activity.

59. A method for protecting cells against cell death, comprising: contacting the cells with a polypeptide having at least 80 percent amino acid sequence identity to the amino acid sequence of a soluble CD14 polypeptide (SEQ ID NO: 5), a fragment thereof, or at least one peptide derived therefrom which retain the protective activity of the complete soluble CD14 polypeptide.

60. The method of claim 59, wherein the at least one CD14 peptide comprises an amino acid sequence of SEQ ID NO: 1, or SEQ ID NO: 3, or any variant thereof.

61. The method of claim 59, wherein the at least one CD14 peptide comprises a combination of at least a first CD14 peptide and a second CD14 peptide.

62. The method of claim 61, wherein the first CD14 peptide comprises the amino acid sequence of SEQ ID NO: 1 and the second CD14 peptide comprises the amino acid sequence of SEQ ID NO: 3, or any variant thereof.

63. The method of claim 59, wherein the cell death is caused by apoptosis and wherein the protective activity is an anti-apoptotic activity.

64. The method of claim 59, wherein the cells are lymphocytes.

65. A method of treating and/or preventing a medical condition associated with cell death, comprising: administering to a subject in need thereof a therapeutically effective amount of a polypeptide having at least 80 percent amino acid sequence identity to the amino acid sequence of a soluble CD14 polypeptide (SEQ ID NO: 5), a fragment thereof, or a CD14 peptide derived therefrom which retain the protective activity of the complete soluble CD14 polypeptide.

66. The method of claim 65, wherein the at least one CD14 peptide comprises an amino acid sequence of SEQ ID NO: 1, or SEQ ID NO: 3, or any variant thereof.

67. The method of claim 65, wherein the at least one CD14 peptide comprises a combination of at least a first CD14 peptide and a second CD14 peptide.

68. The method of claim 67, wherein the first CD14 peptide comprises the amino acid sequence of SEQ ID NO: 1 and the second CD14 peptide comprises the amino acid sequence of SEQ ID NO: 3, or any variant thereof.

69. The method of claim 65, wherein the soluble CD14 or the CD14 peptides are human.

70. The method of claim 65, wherein the medical condition is selected from the group consisting of stem cell transplantation, immune deficiencies, autoimmune diseases, central nervous system (CNS) disorders, heart diseases, infectious diseases, aging, cancer and chemotherapy side effects.

71. A method of determining apoptotic status of cells in a subject, comprising: measuring the amount, or percentage of the total, of cells having internally expressed MO2, whereby MO2 positive cells represent the apoptosis-protected cell population.
